(19) United States
(12) Patent Application Publication
(10) Pub. No.: US 2018/0338795 A1
(43) Pub. Date: Nov. 29, 2018

(54) BREAST X-RAY IMAGING APPARATUS AND PUNCTURE ASSISTANCE SYSTEM

(71) Applicant: Canon Medical Systems Corporation, Otawara-shi (JP)

(72) Inventors: Atsuko SUGIYAMA, Nasushiobara (JP); Yoshimasa KOBAYASHI, Nasushiobara (JP)

(73) Assignee: Canon Medical Systems Corporation, Otawara-shi (JP)

(21) Appl. No.: 15/989,917

(22) Filed: May 25, 2018

(30) Foreign Application Priority Data
May 29, 2017 (JP) 2017-105213

(51) Int. Cl. A61B 34/10; A61B 6/00; A61B 10/00; A61B 10/02; A61B 17/34; A61B 6/02; A61B 6/04; A61B 90/11; A61B 90/17

(52) U.S. Cl. CPC A61B 34/10; A61B 6/502; A61B 10/0041; A61B 10/0275; A61B 17/3403; A61B 2017/3407; A61B 6/025; A61B 6/0414; A61B 90/11; A61B 90/17; A61B 2034/107; A61B 6/5217

(57) ABSTRACT

A breast X-ray imaging apparatus according to an embodiment includes processing circuitry. The processing circuitry collects three-dimensional image data of a breast of a subject. The processing circuitry detects a lesion from the three-dimensional image data. The processing circuitry derives a puncture path for inserting a puncture needle into the breast, by analyzing linearity in three-dimensional distribution of the lesion and based on an analysis result of the linearity.

### CROSS-REFERENCE TO RELATED APPLICATIONS

This application is based upon and claims the benefit of priority from Japanese Patent Application No. 2017-105213, filed on May 29, 2017, the entire contents of which are incorporated herein by reference.

### FIELD

Embodiments described herein relate generally to a breast X-ray imaging apparatus and a puncture assistance system.

### BACKGROUND

Conventionally, a technique for assisting puncture performed during breast biopsy by using image data collected by a breast X-ray imaging apparatus has been known.

For example, a technique of mechanically guiding a puncture position by obtaining the position of a lesion generated in the breast in a three-dimensional manner, using two-dimensional image data such as a scout image and a stereo image captured by a mammography apparatus that is an example of the “breast X-ray imaging apparatus”, and by inputting the obtained position in a puncture device has been known. In recent years, a three-dimensional mammography capable of collecting three-dimensional image data formed of a plurality of sliced images by a single imaging has become popular, and a technique of guiding the puncture position using the three-dimensional image data has also been developed.

### BRIEF DESCRIPTION OF THE DRAWINGS

FIG. 1 is a diagram illustrating a configurational example of a mammography apparatus and a puncture assistance system according to a first embodiment;

FIG. 2 is a diagram illustrating an example of a puncture needle according to the first embodiment;

FIG. 3 is a diagram illustrating an example of how the subject is positioned when puncture is performed using the mammography apparatus according to the first embodiment;

FIG. 4 is a diagram for explaining how a puncture path is derived by a derivation function according to the first embodiment;

FIG. 5 is a diagram for explaining how puncture depth is derived by the derivation function according to the first embodiment;

FIG. 6 is a diagram for explaining how puncture depth is derived by the derivation function according to the first embodiment;

FIG. 7 is a diagram for explaining how puncture depth is derived by the derivation function according to the first embodiment;

FIG. 8 is a diagram illustrating an example of how the puncture path is displayed by the derivation function according to the first embodiment;

FIG. 9 is a diagram illustrating an example of an MIP image of the breast displayed by the derivation function according to the first embodiment; and

FIG. 10 is a flowchart illustrating a processing procedure of puncture assisted by the mammography apparatus according to the first embodiment.

### DETAILED DESCRIPTION

A breast X-ray imaging apparatus according to embodiments includes a collection unit, a detection unit, and a derivation unit. The collection unit collects three-dimensional image data of a breast of a subject. The detection unit detects a lesion from the three-dimensional image data. The derivation unit derives a puncture path for inserting a puncture needle into the breast, by analyzing the linearity in the three-dimensional distribution of a plurality of the lesions, and based on the analysis result of the linearity.

Hereinafter, embodiments of a breast X-ray imaging apparatus and a puncture assistance system will be described in detail with reference to the accompanying drawings. It is to be noted that in the following embodiments, the breast X-ray imaging apparatus disclosed in the present application is applied to a mammography apparatus.

### First Embodiment

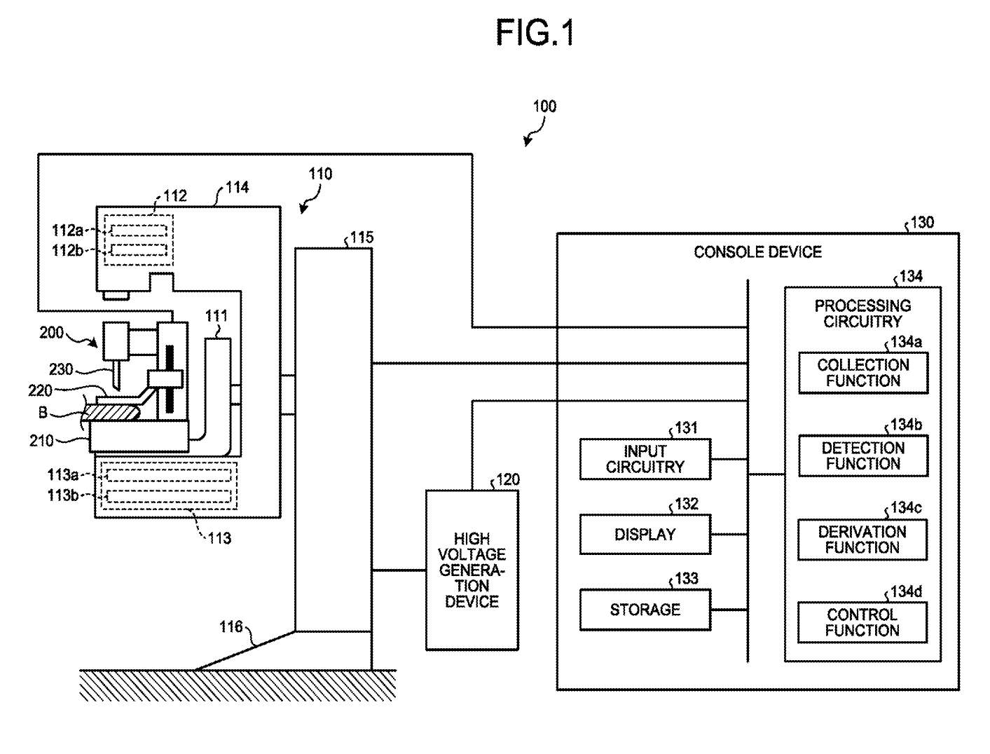

FIG. 1 is a diagram illustrating a configurational example of a mammography apparatus and a puncture assistance system according to a first embodiment. For example, as illustrated in FIG. 1, a mammography apparatus 100 includes an imaging stand device 110, a high voltage generation device 120, and a console device 130.

The imaging stand device 110 includes a support table 111, an X-ray generation device 112, an X-ray detection device 113, an imaging stand 114, a stand 115, and a base 116.

The support table 111 is a table for supporting a breast B of a subject.

The X-ray generation device 112 includes an X-ray tube 112a and an X-ray diaphragm 112b. The X-ray tube 112a generates X-rays. The X-ray diaphragm 112b controls the irradiation range of X-rays generated from the X-ray tube 112a.

The X-ray detection device 113 includes an X-ray detection unit 113a and a signal processing circuitry 113b. The X-ray detection unit 113a detects the X-rays transmitted through the breast B and the support table 111, and converts the X-rays into electric signals. The signal processing circuitry 113b generates X-ray projection data from the electric signals converted by the X-ray detection unit 113a.

The imaging stand 114 supports the X-ray generation device 112, the support table 111, and the X-ray detection device 113. More specifically, the imaging stand 114 supports the X-ray generation device 112 and the X-ray detection device 113 in a state in which the X-ray generation device 112 and the X-ray detection device 113 face each other with a rotation axis in the horizontal direction interposed therebetween. The imaging stand 114 movably supports the support table 111 between the X-ray generation device 112 and the X-ray detection device 113. The imaging stand 114 moves the support table 111 according to an operation of an operator.

The stand 115 supports the imaging stand 114. More specifically, the stand 115 not only movably supports the imaging stand 114 in the vertical direction with the rotation axis described above, but also rotatably supports the imaging stand 114 around the rotation axis described above. The stand 115 moves or rotates the imaging stand 114 according to an operation of the operator.

The base 116 supports the stand 115 in an upright manner.

The high voltage generation device 120 is connected to the X-ray tube 112a, and supplies high voltage so that the X-ray tube 112a can generate X-rays.

The console device 130 controls the whole mammography apparatus 100. More specifically, the console device 130 includes input circuitry 131, a display 132, a storage 133, and processing circuitry 134.

The input circuitry 131 receives input operations of various instructions and various types of information from the operator. More specifically, the input circuitry 131 is connected to the processing circuitry 134, converts the input operations received from the operator into electric signals, and outputs the electric signals to the processing circuitry 134. For example, the input circuitry 131 is implemented by a track ball, a switch button, a mouse, a keyboard, a touch panel, and the like.

The display 132 displays various types of information and various images. More specifically, the display 132 is connected to the processing circuitry 134, and converts data on various types of information and various images sent from the processing circuitry 134 into display electric signals for output. For example, the display 132 is implemented by a liquid crystal monitor, a cathode ray tube (CRT) monitor, a touch panel, and the like.

The storage 133 stores therein various types of data. More specifically, the storage 133 stores therein X-ray projection data generated by the signal processing circuitry 113b, image data generated by the processing circuitry 134, and the like. For example, the storage 133 is implemented by a semiconductor memory device such as a random access memory (RAM) and a flash memory, a hard disk, an optical disk, and the like.

The processing circuitry 134 controls the entire mammography apparatus 100 by controlling the components included in the mammography apparatus 100. For example, the processing circuitry 134 is implemented by a processor.

With such a configuration, the mammography apparatus 100 according to the present embodiment has a configuration for assisting puncture performed during biopsy of the breast B of the subject. For example, the mammography apparatus 100 according to the present embodiment is used for precise breast cancer examination and the like. In the following, it is assumed that the lesion sampled by the puncture is a calcification.

The mammography apparatus 100 according to the present embodiment exposes the breast B of the subject with X-rays while moving the X-ray tube 112a along a predetermined scanning path, and detects the X-rays incident on the detection surface of the X-ray detection unit 113a. The mammography apparatus 100 according to the present embodiment also generates an X-ray image of each frame based on the detected X-rays. In other words, the mammography apparatus 100 according to the present embodiment has a configuration of an X-ray tomographic apparatus (may also be referred to as a tomosynthesis).

The mammography apparatus 100 according to the present embodiment also includes a puncture adapter 200. The puncture adapter 200 inserts a puncture needle into the breast B of the subject under the control of the console device 130. In this example, the puncture adapter 200 can respond to various puncture techniques such as vacuum assistant biopsy and core needle biopsy.

More specifically, the puncture adapter 200 includes an adapter support table 210, an adapter compression plate 220, and a puncture needle 230.

The adapter support table 210 supports the breast B when the breast B of the subject is to be punctured.

The adapter compression plate 220 fixes the breast B placed on the adapter support table 210 onto the adapter support table 210, by compressing and spreading the breast B.

More specifically, the adapter compression plate 220 is movably provided in a direction approaching the adapter support table 210 and in a direction away from the adapter support table 210, and moves in both directions according to an operation of the operator. In this example, an access hole penetrating from the front surface toward the rear surface of the plate is formed on a part of the adapter compression plate 220. The puncture needle 230 is inserted into the breast B through the access hole.

The puncture needle 230 is a needle for sampling a specimen from the breast B.

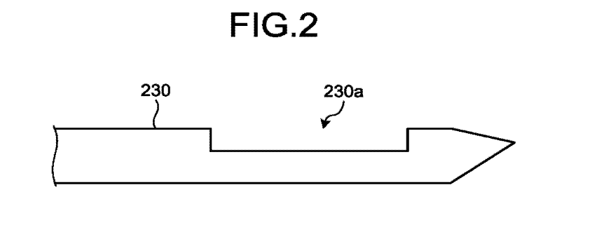

FIG. 2 is a diagram illustrating an example of the puncture needle 230 according to the first embodiment. For example, as illustrated in FIG. 2, the tip end of the puncture needle 230 has a sharp-pointed shape, and is inserted into the breast B of the subject from the tip end. Moreover, the puncture needle 230 is formed in a hollow cylindrical shape, and has an opening 230a communicating from the outside toward the inside of the puncture needle 230 at a part of the side surface. The opening 230a is a hole for sampling a specimen when the puncture needle 230 is inserted into the breast B.

In the present embodiment, the puncture adapter 200 is installed on the support table 111 of the imaging stand device 110 so that the puncture adapter 200 can be disposed between the X-ray generation device 112 and the X-ray detection device 113. Consequently, the mammography apparatus 100 according to the present embodiment can take an image of the breast B while the breast B is fixed on the adapter support table 210.

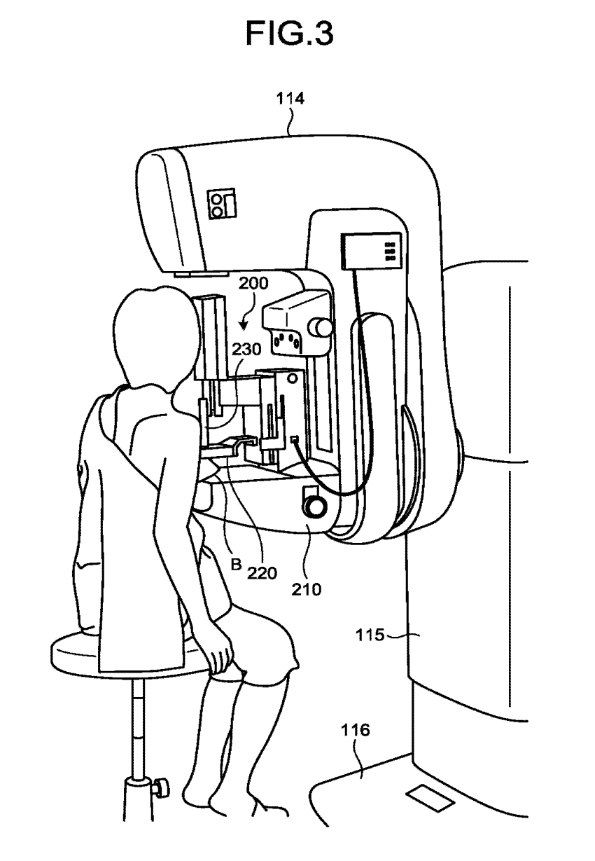

FIG. 3 is a diagram illustrating an example of how the subject is positioned when puncture is performed using the mammography apparatus 100 according to the first embodiment. For example, as illustrated in FIG. 3, to puncture the breast B, the subject will place the breast B on the adapter support table 210 while the subject is seated on a chair or the like in a sitting-up position with raised upper half body. When the adapter compression plate 220 is moved in the direction approaching the adapter support table 210, the breast B placed on the adapter support table 210 is fixed on the adapter support table 210, while the breast B is being compressed and spread. The posture of the subject during puncture is not limited to the sitting-up position, but may also be a side-lying position, or a prone position.

In the present embodiment, the processing circuitry 134 of the console device 130 mechanically guides the puncture of the breast B, by controlling the puncture adapter 200 using the three-dimensional image data that is a captured image of the breast B of the subject.

In this process, for example, to guide the puncture using the three-dimensional image data, there is also a method of specifying a puncture target position to the puncture adapter 200 using a dot. For example, when z is the position of each sliced image in the alignment direction of a plurality of sliced images, and (x, y) is the two-dimensional position in each of the sliced images in the three-dimensional image data, the dot of the puncture target position is specified by three-dimensional positions (x, y, z) obtained by combining z and (x, y).

However, when the puncture target position is specified by a dot, a plurality of the calcifications around the dot are not always sufficiently included in the specimen, depending on the angle by which the puncture needle 230 is inserted. In general, the specimen sampled by the puncture is required to include not only the calcification at the target position, but also a sufficient amount of calcifications around the target position. Whether the sampled specimen includes a sufficient amount of calcifications is confirmed by X-ray imaging or the like after the puncture. As a result, if it is determined that the calcifications are not sufficiently included, the puncture may need to be performed again, which largely burdens the subject.

Thus, the mammography apparatus 100 according to the present embodiment is configured to be able to sample a specimen containing a lesion with more certainty.

More specifically, in the present embodiment, the processing circuitry 134 includes a collection function 134a, a detection function 134b, a derivation function 134c, and a control function 134d. In the present embodiment, the puncture adapter 200 and the detection function 134b, the derivation function 134c, and the control function 134d of the processing circuitry 134 configure a puncture assistance system.

In this example, the collection function 134a is an example of a collection unit in the appended claims. The detection function 134b is an example of a detection unit in the appended claims. The derivation function 134c is an example of a derivation unit in the appended claims. The control function 134d is an example of a control unit in the appended claims.

The collection function 134a collects three-dimensional image data of the breast B of the subject.

More specifically, the collection function 134a executes three-dimensional mammography by controlling the high voltage generation device 120 and the X-ray generation device 112. More specifically, the collection function 134a irradiates the breast B of the subject with X-rays from a plurality of different directions, while moving the X-ray tube 112a along a predetermined scanning path. The collection function 134a also generates an X-ray image of each frame based on the X-rays detected by the X-ray detection unit 113a. The collection function 134a generates three-dimensional image data (may also be referred to as volume data) based on the generated X-ray image. For example, the collection function 134a generates three-dimensional image data obtained by capturing an image of the breast B in the mediolateral-oblique (MLO) direction and three-dimensional image data obtained by capturing an image of the breast B in the cranio-caudal (CC) direction. The collection function 134a stores the generated three-dimensional image data in the storage 133.

The detection function 134b detects calcifications from the three-dimensional image data collected by the collection function 134a.

More specifically, the detection function 134b reads out the three-dimensional image data of the breast B collected by the collection function 134a from the storage 133, and detects calcifications from the read three-dimensional image data. In this example, the detection function 134b may use various known methods for detecting calcifications. For example, the detection function 134b detects calcifications by extracting a region where the luminance value is equal to or more than a threshold, based on the luminance value of each voxel included in the three-dimensional image data.

The derivation function 134c derives the puncture path for inserting the puncture needle 230 into the breast B, by analyzing the linearity in the three-dimensional distribution of calcifications detected by the detection function 134b, and based on the analysis results of the linearity. The derivation function 134c derives center of gravity of the three-dimensional distribution of calcifications, and derives the puncture depth for inserting the puncture needle 230, based on the position of the center of gravity and the position of the opening 230a of the puncture needle 230.

More specifically, the derivation function 134c disposes the calcifications detected by the detection function 134b, on the three-dimensional coordinate indicating an imaging space to be imaged by the mammography apparatus 100. The three-dimensional coordinate in this example is defined in advance based on the position of the X-ray detection unit 113a and the like, as a coordinate specific to the mammography apparatus 100. The derivation function 134c then derives the puncture path by analyzing the linearity of the distribution of calcifications disposed on the three-dimensional coordinate by performing a statistical process, and based on the analysis results of the linearity.

In the present embodiment, the derivation function 134c derives the regression line based on the distribution of calcifications disposed on the three-dimensional coordinate, and sets the derived regression line as the puncture path.

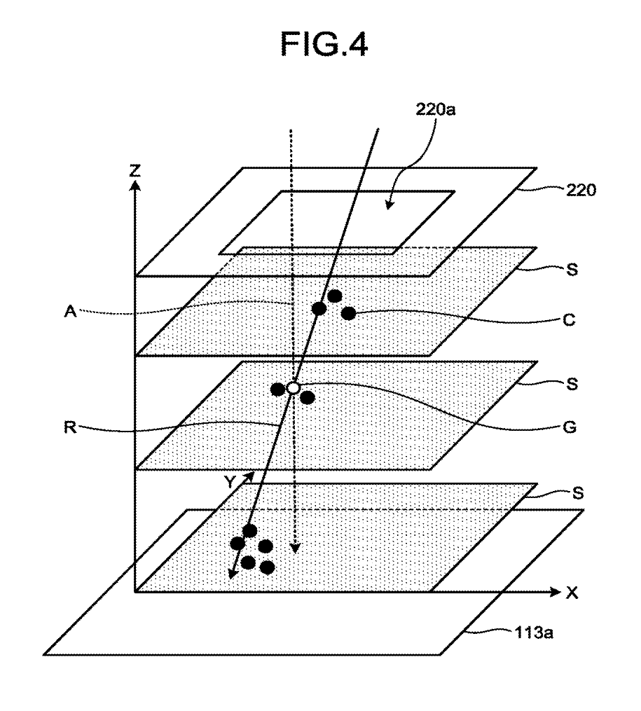

FIG. 4 is a diagram for explaining how a puncture path is derived by the derivation function 134c according to the first embodiment. In the example illustrated in FIG. 4, the three-dimensional coordinate is defined by the X-axis and the Y-axis along the two-dimensional direction on the detection surface of the X-ray detection unit 113a, and the Z-axis perpendicular to the detection surface of the X-ray detection unit 113a. Moreover, in the example illustrated in FIG. 4, the positions of a plurality of sliced images S, the position of the X-ray detection unit 113a, the position of the adapter compression plate 220, and the position of an access hole 220a formed on the adapter compression plate 220 included in the three-dimensional image data are indicated in the three-dimensional coordinate.

For example, as illustrated in FIG. 4, the derivation function 134c derives a regression line R from the distribution of a plurality of calcifications C disposed on the three-dimensional coordinate. Moreover, the derivation function 134c derives the center of gravity G of the distribution of the calcifications C disposed on the three-dimensional coordinate. The derivation function 134c then sets the puncture path based on the derived regression line R.

For example, when the puncture target position is specified by a dot as the path illustrated by a broken line A in FIG. 4, the puncture path may be set without including the calcifications around the target position. In this case, the sampled specimen may not include a sufficient amount of calcifications. However, in the present embodiment, the puncture path is set based on the regression line R derived from the distribution of calcifications. Thus, it is possible to set the puncture path so as to include calcifications as much as possible. Consequently, it is possible to include calcifications in the sampled specimen with more certainty.

The puncture directions (directions of inserting the puncture needle 230) when the breast B is to be punctured include a direction from the upper side toward the lower side, a direction from the right side toward the left side, a direction from the left side toward the right side, and a direction from the front side toward the depth side with respect to the breast B. In general, the limitations (access limitations) on the device and the inspection while the puncture needle 230 is to be inserted into the breast B differ according to each direction.

In the present embodiment, the derivation function 134c derives the puncture path and the puncture depth according to the puncture direction and by taking into account the limitations on the device and inspection.

For example, when the breast B is punctured in the direction from the upper side toward the lower side, the puncture needle 230 is inserted into the breast B through the access hole 220a formed on the adapter compression plate 220. Thus, for example, when the regression line derived from the distribution of calcifications is from the upper side toward the lower side, the derivation function 134c derives a path that is closest to the regression line, that passes through the center of gravity G, and that passes through the access hole 220a as the puncture path. In this case, it is assumed that the position and size of the access hole 220a are set in advance in association with the three-dimensional coordinate, depending on the state of device at the time of puncture.

Moreover, for example, when the breast B is punctured in the direction from the right side toward the left side, the direction from the left side toward the right side, and the direction from the front side toward the depth side, the puncture needle 230 is inserted into the breast B by avoiding the adapter support table 210 and the adapter compression plate 220. Thus, for example, when the regression line derived from the distribution of calcifications is the line from the right side toward the left side, the line from the left side toward the right side, or the line from the front side toward the depth side, the derivation function 134c derives a path that is closest to the regression line, that passes through the center of gravity G, and that does not intersect with the adapter support table 210 and the adapter compression plate 220 as the puncture path. In this case, it is assumed that the position and size of the adapter support table 210 and the adapter compression plate 220 are set in advance in association with the three-dimensional coordinate, depending on the state of the device at the time of puncture.

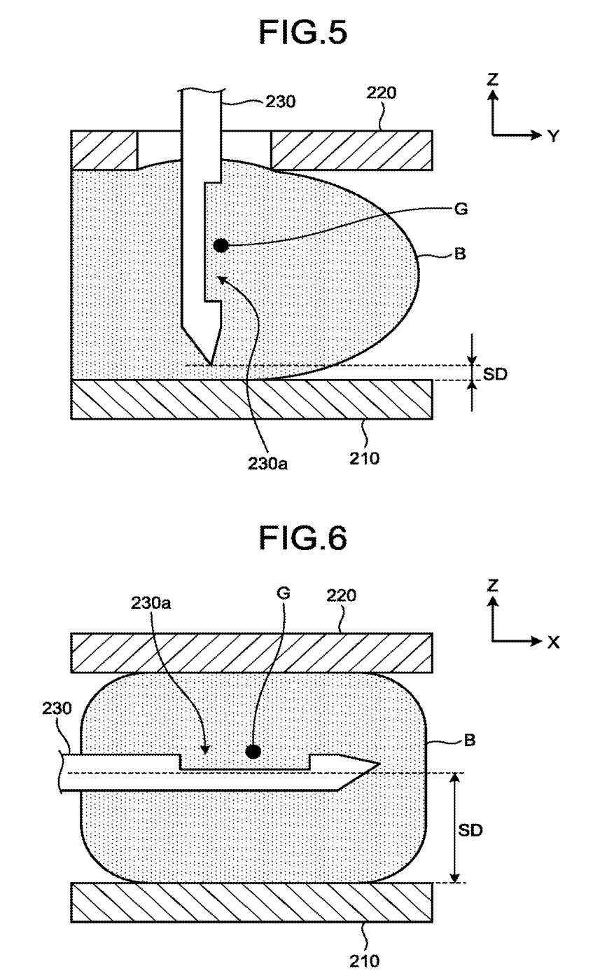

FIGS. 5 to 7 are diagrams for explaining how the puncture depth is derived by the derivation function 134c according to the first embodiment. For example, as illustrated in FIGS. 5 to 7, the derivation function 134c derives the puncture depth of the puncture needle 230 so that the center position of the opening 230a of the puncture needle 230 in the axis direction and the position of the center of gravity G of the distribution of calcifications can be matched with each other. For example, the puncture depth is distance from the skin surface of the breast B to the tip end of the puncture needle 230.

For example, as illustrated in FIG. 5, when the breast B is punctured in the direction from the upper side toward the lower side (vertical approach), a predetermined distance (hereinafter, referred to as safety distance) SD needs to be secured between the tip end of the puncture needle 230 and the adapter support table 210 so that the tip end of the puncture needle 230 will not penetrate through the breast B. Thus, in this case, for example, the derivation function 134c derives the puncture depth (depth in the Z-axis direction) of the puncture needle 230 so that the distance between the tip end of the puncture needle 230 and the adapter support table 210 can be equal to or more than the safety distance SD, and the center position of the opening 230a in the axis direction can be closest to the position of the center of gravity G of the distribution of calcifications. In this process, for example, when the center of gravity G is not included within the range of the opening 230a of the puncture needle 230, when the safety distance SD is secured between the tip end of the puncture needle 230 and the adapter support table 210, the operator may be notified.

Moreover, for example, as illustrated in FIG. 6, when the breast B is punctured in the direction from the left side toward the right side (lateral approach), the safety distance SD needs to be secured between the axial center of the puncture needle 230 and the adapter support table 210. Thus, in this case, for example, the derivation function 134c derives the position (position in the Z-axis direction) of the puncture path of the puncture needle 230 so that the distance between the axial center of the puncture needle 230 and the adapter support table 210 can be equal to or more than the safety distance SD. In this process, for example, when the center of gravity G is not included within the range of the opening 230a of the puncture needle 230, when the safety distance SD is secured between the axial center of the puncture needle 230 and the adapter support table 210, the operator may be notified.

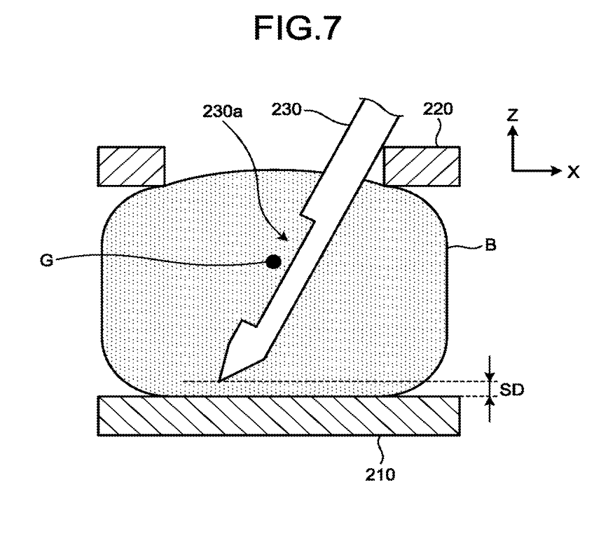

Moreover, for example, as illustrated in FIG. 7, the breast B may be punctured obliquely (oblique access). In this case, for example, the derivation function 134c derives the puncture depth (depth in the Z-axis direction) of the puncture needle 230, the position of the puncture path (position in the Z-axis direction), and the inclination (inclination with respect to the Z-axis direction) so that the distance between the tip end of the puncture needle 230 and the adapter support table 210 can be equal to or more than the safety distance SD, and the center position of the opening 230a in the axis direction can be closest to the position of the center of gravity G of the distribution of calcifications.

The derivation function 134c then displays the derived information on the puncture path on the display 132.

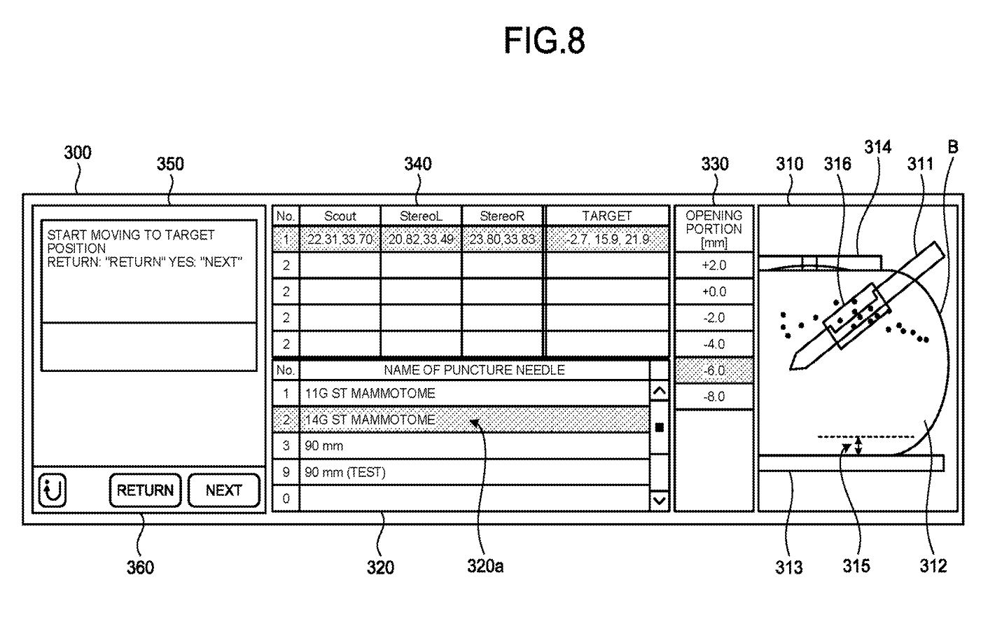

FIG. 8 is a diagram illustrating an example of how the puncture path is displayed by the derivation function 134c according to the first embodiment. For example, as illustrated in FIG. 8, the derivation function 134c displays an operation screen 300 on which various graphical user interfaces (GUIs) are disposed on the display 132.

For example, the operation screen 300 includes an area 310 for displaying information on the puncture path, an area 320 for displaying information on the puncture needle 230, an area 330 for displaying information on the opening 230a of the puncture needle 230, an area 340 for displaying information on the puncture target position, an area 350 for displaying a message for the operator, an area 360 for displaying a button and an icon for receiving various operations from the operator, and the like.

The derivation function 134c displays a graphic 311 indicating the puncture path and the puncture depth in the area 310 for displaying information on the puncture path. For example, the derivation function 134c generates a maximum intensity projection (MIP) image 312 of the breast B and displays the information on the area 310 for displaying information on the puncture path.

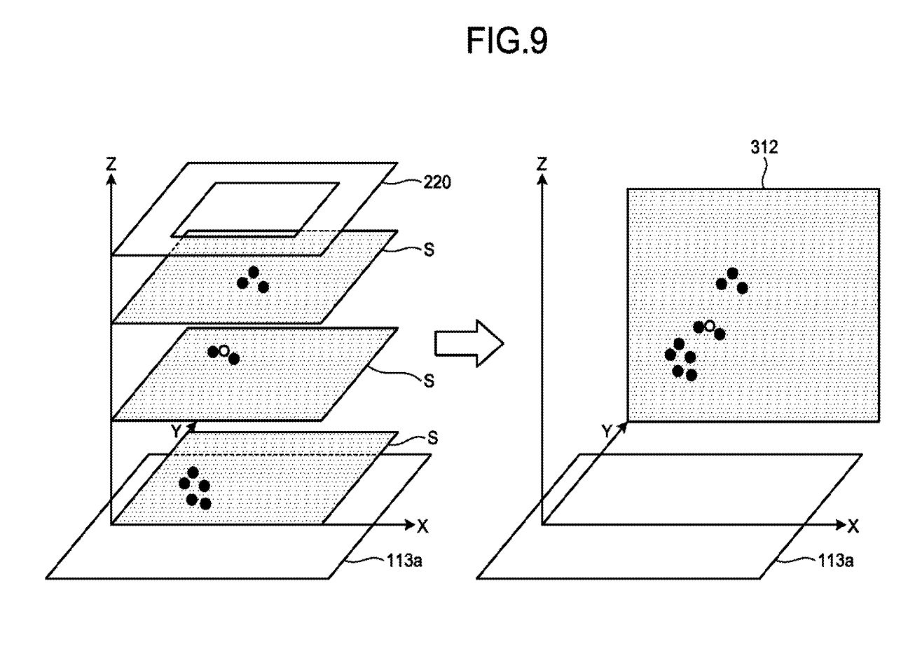

FIG. 9 is a diagram illustrating an example of the MIP image 312 of the breast B displayed by the derivation function 134c according to the first embodiment. For example, as illustrated in FIG. 9, the derivation function 134c generates the MIP image 312 on which the three-dimensional image data is projected in the Y-axis direction. In this process, distribution of calcifications generated in the breast B will be projected on the generated MIP image 312.

The MIP image 312 generated by the derivation function 134c may be an image projected in the Z-axis direction or an image projected in the X-axis direction. For example, the derivation function 134c generates the MIP image 312 in the projection direction so that the puncture path can be easily recognized, according to the direction of the derived puncture path.

Then, for example, as illustrated in FIG. 8, the derivation function 134c displays the graphic 311 simulating the shape of the puncture needle 230 by overlapping the graphic 311 on the generated MIP image 312. In this process, the derivation function 134c displays the graphic 311 by aligning the graphic 311 with the MIP image 312 so that the positions of the derived puncture path and the puncture depth can be indicated based on the three-dimensional coordinate.

In this process, for example, the derivation function 134c may also display a graphic 313 indicating the position of the adapter support table 210, a graphic 314 indicating the position of the adapter compression plate 220, and a graphic 315 indicating the safety distance in an overlapping manner with the MIP image 312, in addition to the graphic 311 indicating the puncture path and the puncture depth. In this process, the derivation function 134c displays the graphics by aligning each of the graphics with the MIP image 312 based on the three-dimensional coordinate.

The derivation function 134c may also display a sliced image generated from the three-dimensional image data instead of displaying the MIP image 312. In this case, for example, the derivation function 134c generates and displays a sliced image at the position where the derived puncture path passes through.

The derivation function 134c also estimates the thickness of the puncture needle 230 based on the puncture path and the distribution range of calcifications.

More specifically, the derivation function 134c estimates the thickness of the puncture needle 230 suitable for performing puncture using the derived puncture path, based on the distribution of calcifications detected from the three-dimensional image data. For example, the derivation function 134c selects suitable thickness from a plurality of thicknesses of the puncture needle 230 stored in the storage 133 in advance, based on the size of the distribution range of calcifications in the direction perpendicular to the puncture path.

In this case, for example, a plurality of thresholds are set in a stepwise manner with respect to a plurality of the sizes of the distribution range of calcifications in the direction perpendicular to the puncture path, and the puncture needle 230 with a suitable thickness is associated with each of the thresholds. The derivation function 134c selects the puncture needle 230 of a suitable thickness from a plurality of the puncture needles 230, by comparing the size of the distribution range of calcifications in the direction perpendicular to the puncture path with each of the thresholds.

Then, for example, as illustrated in FIG. 8, the derivation function 134c displays information on the puncture needles 230 in the area 320 for displaying information on the puncture needle 230, and also displays information 320a indicating the selected puncture needle 230 in the area 320 in an identifiable manner. Thus, the derivation function 134c can present a recommended puncture needle 230 to the operator from the puncture needles 230 prepared in advance, when the puncture is performed through the derived puncture path.

Then, the derivation function 134c receives an operation of selecting one puncture needle 230 from the displayed puncture needles 230 from the operator. For example, the derivation function 134c may dynamically change the thickness of the graphic 311 indicating the puncture path and the puncture depth, depending on the thickness of the puncture needle 230 selected by the operator.

Furthermore, the derivation function 134c estimates a region where a specimen is to be sampled by the puncture needle 230, and displays information indicating the position and size of the region in association with the puncture path.

For example, as illustrated in FIG. 8, the derivation function 134c displays a graphic 316 indicating a range of region where a specimen is to be sampled by the puncture needle 230, by overlapping the graphic 316 onto the graphic 311 indicating the puncture path and the puncture depth. In this case, for example, information on the position and size of the region where a specimen is to be sampled by each of the puncture needles 230 is stored in the storage 133 in advance. The derivation function 134c then acquires the position and size of the region corresponding to the puncture needle 230 of the estimated thickness or the puncture needle 230 selected by the operator by referring to the storage 133, and determines the position and size when the graphic 311 is to be displayed.

In this process, for example, the derivation function 134c displays information on the region relating to the puncture needle 230, at the point when the puncture needle 230 used for puncture is finally determined. Alternatively, for example, the derivation function 134c may dynamically display information on the region relating to the selected puncture needle 230, every time the operator selects the puncture needle 230.

The derivation function 134c then corrects the puncture path according to an operation received from the operator.

The derivation function 134c then receives an operation of changing the position of the graphic 311 indicating the puncture path and the puncture depth from the operator through the operation screen 300, and corrects the puncture path set in advance according to the received operation. Consequently, the operator can suitably adjust the puncture path and the puncture depth.

The control function 134d controls the puncture adapter 200 based on the puncture path derived by the derivation function 134c.

More specifically, the control function 134d receives the instruction to execute puncture from the operator through the operation screen 300. Upon receiving the instruction to execute puncture from the operator, the control function 134d controls the puncture adapter 200 so that the puncture can be performed in the puncture path, based on the puncture path (the puncture path derived by the derivation function 134c or the puncture path corrected by the operator) set at that point. In this process, for example, the control function 134d controls the puncture adapter 200 by entering the information indicating the position and direction of the puncture path in the puncture adapter 200.

As described above, the processing functions of the processing circuitry 134 have been described. In the example illustrated in FIG. 1, each of the processing functions of the processing circuitry 134 is implemented by a single processing circuitry. However, the embodiment is not limited thereto. The processing functions of the processing circuitry 134 may be implemented in a manner that the processing functions are suitably dispersed or integrated in a single or a plurality of the processing circuitries.

For example, the processing functions of the processing circuitry 134 are stored in the storage 133 in the form of a computer-executable program. The processing circuitry 134 implements the processing functions corresponding to computer programs, by reading out the computer programs from the storage 133, and executing the read computer programs. In other words, the processing circuitry 134 that has read out the computer programs has the processing functions illustrated in FIG. 1.

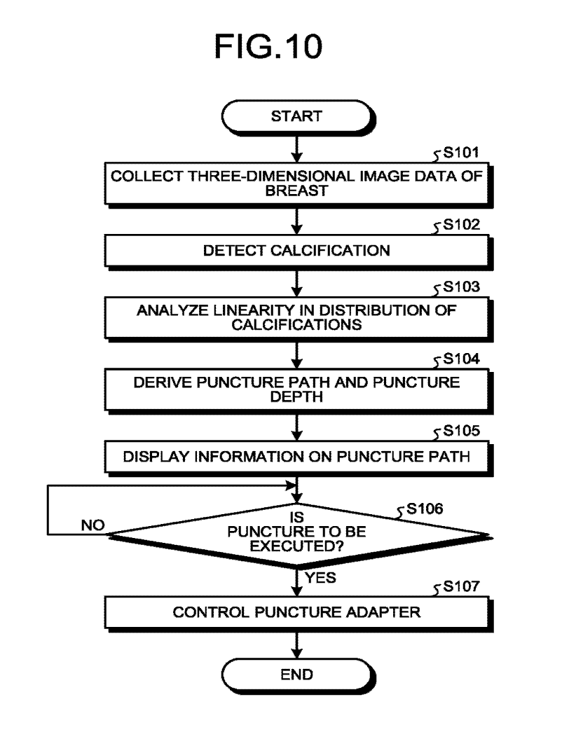

FIG. 10 is a flowchart illustrating a processing procedure of puncture assisted by the mammography apparatus 100 according to the first embodiment. For example, as illustrated in FIG. 10, in the present embodiment, the collection function 134a first collects three-dimensional image data on the breast B of the subject (step S101). In this process, for example, step S101 is implemented when the processing circuitry 134 reads out a predetermined computer program corresponding to the collection function 134a from the storage 133 and executes the computer program.

Subsequently, the detection function 134b detects calcifications from the three-dimensional image data collected by the collection function 134a (step S102). In this process, for example, step S102 is implemented when the processing circuitry 134 reads out a predetermined computer program corresponding to the detection function 134b from the storage 133 and executes the computer program.

Subsequently, the derivation function 134c analyzes the linearity in the three-dimensional distribution of calcifications detected by the detection function 134b (step S103), and derives the puncture path and the puncture depth (step S104). The derivation function 134c then displays the information on the puncture path on the display 132 (step S105). Steps S103 to S105 are implemented, for example, when the processing circuitry 134 reads out a predetermined computer program corresponding to the derivation function 134c from the storage 133 and executes the computer program.

When an instruction to execute the puncture is received from the operator (Yes at step S106), the control function 134d controls the puncture adapter 200 (step S107) so that the puncture can be performed in the puncture path set at that point. In this process, for example, steps S106 and S107 are implemented when the processing circuitry 134 reads out a predetermined computer program corresponding to the control function 134d from the storage 133 and executes the computer program.

As described above, in the present embodiment, the puncture path for inserting the puncture needle into the breast is derived based on the analysis result obtained by analyzing the linearity in the three-dimensional distribution of calcifications. Thus, it is possible to set the puncture path so as to include calcifications as much as possible. Consequently, with the present embodiment, it is possible to sample a specimen containing a lesion with more certainty. As a result, it is possible to reduce rebiopsy.

In the first embodiment described above, a part of the processing functions of the processing circuitry 134 can be performed by suitably changing the processing functions. In the following, modifications according to the first embodiment described above will be described as the other embodiments.

### Second Embodiment

For example, in the embodiment described above, the derivation function 134c derives a single puncture path. However, the embodiment is not limited thereto.

As described above, the puncture directions (directions of inserting the puncture needle 230) when the breast B is to be punctured include the direction from the upper side toward the lower side, the direction from the right side toward the left side, the direction from the left side toward the right side, and the direction from the front side toward the depth side with respect to the breast B. Then, for example, as a second embodiment, the derivation function 134c may derive the puncture path for each of these directions.

In this case, for example, the derivation function 134c derives a path that is closest to the regression line derived from the distribution of calcifications, that passes through the center of gravity G, and that passes through the access hole 220a as the puncture path, for the direction from the upper side toward the lower side with respect to the breast B. Moreover, for example, the derivation function 134c derives a path that is closest to the regression line derived from the distribution of calcifications, that passes through the center of gravity G, and that accesses the breast B from the right side as the puncture path, for the direction from the right side toward the left side with respect to the breast B. Furthermore, for example, the derivation function 134c derives a path that is closest to the regression line derived from the distribution of calcifications, that passes through the center of gravity G, and that accesses the breast B from the left side as the puncture path, for the direction from the left side toward the right side with respect to the breast B. Still furthermore, for example, the derivation function 134c derives a path that is closest to the regression line derived from the distribution of calcifications, that passes through the center of gravity G, and that accesses the breast B from the front side as the puncture path, for the direction from the front side toward the depth side with respect to the breast B. In the present embodiment, similar to the first embodiment, the derivation function 134c derives the puncture path and the puncture depth by taking into account the limitations on the device and inspection according to the puncture direction.

Moreover, the derivation function 134c displays the graphic 311 indicating the puncture path and the puncture depth in the area 310 for displaying information on the puncture path, for each of a plurality of the derived puncture paths. In this process, the derivation function 134c may simultaneously display a plurality of the graphics 311 or may switchingly display the graphics 311 according to an operation of the operator. The derivation function 134c then receives an operation of selecting one puncture path from the displayed puncture paths from the operator, and sets the selected puncture path as the puncture path to be input in the puncture adapter 200.

For example, the derivation function 134c may also receive from the operator an operation of specifying the direction of puncture. In this case, the derivation function 134c derives the puncture path for the direction of puncture received from the operator. Moreover, for example, the derivation function 134c may omit the specification of the direction of puncture. In this case, the derivation function 134c derives the puncture path of the specified direction when the direction of puncture is specified by the operator, and derives the puncture paths of the directions when the direction of puncture is not specified by the operator.

As described above, the postures of the subject when puncture is to be performed include the sitting-up position, the side-lying position, the prone position, and the like. However, the puncture path toward which the puncture needle 230 can be easily inserted differ depending on the posture of the subject. With the present embodiment, it is possible to set a more suitable puncture path depending on the posture of the subject and the like, because the derivation function 134c derives the puncture paths of the plurality of directions.

### Third Embodiment

Moreover, in the first embodiment described above, the derivation function 134c derives the puncture depth for inserting the puncture needle 230 based on the position of center of gravity of the three-dimensional distribution of calcifications and the position of the opening 230a of the puncture needle 230. However, the embodiment is not limited thereto.

For example, as a third embodiment, the derivation function 134c may receive an operation for specifying a region of interest of the breast B from the operator, and derive the puncture depth for inserting the puncture needle 230, based on the position of the region of interest and the position of the opening 230a of the puncture needle 230.

In this case, for example, the derivation function 134c derives the puncture depth of the puncture needle 230 so that the center position of the opening 230a of the puncture needle 230 in the axis direction and the position of the center point of the region of interest specified by the operator can be matched with each other. In the present embodiment, similar to the first embodiment, the derivation function 134c derives the puncture path and the puncture depth by taking into account the limitations on the device and inspection according to the puncture direction.

In the present embodiment, the derivation function 134c does not necessarily derive the center of gravity G of the distribution of calcifications, or may derive the center of gravity G of the distribution of calcifications as in the first embodiment. To derive the center of gravity G of the distribution of calcifications, the derivation function 134c first derives the puncture path and the puncture depth based on the derived center of gravity G, as in the first embodiment. The derivation function 134c then receives an operation for changing the position of the region of interest from the operator, by setting the derived center of gravity G as the region of interest. When the position of the region of interest is changed, the derivation function 134c derives the puncture path and the puncture depth again, based on the changed position of the region of interest.

### Fourth Embodiment

In the first embodiment described above, the derivation function 134c displays the information on the puncture path on the display 132 of the console device 130. However, the embodiment is not limited thereto.

For example, the puncture adapter 200 may include a display as a fourth embodiment. In this case, the derivation function 134c may display the information on the puncture path on the display of the puncture adapter 200. By displaying the information on the puncture path on the display of the puncture adapter 200 in this manner, the operator can confirm the puncture path at the position closer to the breast B. Consequently, the operator can more easily grasp the positional relation between the breast B and the puncture path.

The term “processor” used in the embodiments described above means a central processing unit (CPU), a graphics processing unit (GPU), or a circuit such as an application specific integrated circuit (ASIC), a programmable logic device (for example, a simple programmable logic device (SPLD), a complex programmable logic device (CPLD), and a field programmable gate array (FPGA)), and the like. In this example, a computer program may be directly incorporated in the circuit of the processor, instead of storing a computer program in the storage 133. In this case, the function is implemented when the processor reads and executes the computer program incorporated in the circuit. Each of a plurality of the processors of the present embodiment is not only configured as a single circuit per processor, but may be configured as a single processor by combining a plurality of independent circuits to implement the functions.

For example, the computer program to be executed by the processor is incorporated into a read only memory (ROM), a storage, and the like in advance. The computer programs may also be recorded on a computer-readable storage medium such as a compact disc-read only memory (CD-ROM), a flexible disk (FD), a compact disc-recordable (CD-R), and a digital versatile disc (DVD) in an installable or executable file format in the device. The computer program can also be stored on a computer connected to a network such as the Internet, and can be provided or distributed by being downloaded via the network. For example, the computer program is composed of modules including various functions, which will be described below. As actual hardware, the CPU reads out and executes the computer program from the storage medium such as the ROM, thereby loading and generating the modules on the main memory.

With at least one of the embodiments described above, it is possible to sample a specimen containing a lesion with more certainty.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A breast X-ray imaging apparatus, comprising:
processing circuitry configured to
collect three-dimensional image data of a breast of a subject;
detect a lesion from the three-dimensional image data; and
derive a puncture path for inserting a puncture needle into the breast, by analyzing linearity in three-dimensional distribution of the lesion and based on an analysis result of the linearity.

2. The breast X-ray imaging apparatus according to claim 1, wherein the processing circuitry corrects the puncture path according to an operation received from an operator.

3. The breast X-ray imaging apparatus according to claim 1, wherein the processing circuitry estimates thickness of the puncture needle based on the puncture path and a distribution range of the lesion.

4. The breast X-ray imaging apparatus according to claim 1, wherein
the puncture needle has an opening for sampling a specimen, and
the processing circuitry derives center of gravity of the three-dimensional distribution of the lesion, and further derives puncture depth for inserting the puncture needle based on a position of the center of gravity and a position of the opening of the puncture needle.

5. The breast X-ray imaging apparatus according to claim 1, wherein
the puncture needle has an opening for sampling a specimen, and
the processing circuitry receives an operation of specifying an region of interest of the breast from the operator, and also derives puncture depth for inserting the puncture needle based on a position of the region of interest and a position of the opening of the puncture needle.

6. The breast X-ray imaging apparatus according to claim 1, wherein the processing circuitry estimates a region where a specimen is to be sampled by the puncture needle, and displays information indicating a position and size of the region in association with the puncture path.

7. A puncture assistance system, comprising:
a puncture adapter configured to insert a puncture needle into a breast of a subject; and
processing circuitry configured to
detect a lesion from three-dimensional image data of the breast collected by a breast X-ray imaging apparatus;
derive a puncture path for inserting the puncture needle into the breast, by analyzing linearity in three-dimensional distribution of the lesion and based on an analysis result of the linearity; and
control the puncture adapter, based on the puncture path.
